(19) United States
(12) Patent Application Publication
(10) Pub. No.: US 2023/0285309 A1
(43) Pub. Date: Sep. 14, 2023

(54) METHOD OF PRESERVING A REACTIVE ACTIVE COMPOUND, CAPSULE AND FORMULATION

(71) Applicant: IAMFLUIDICS HOLDING B.V., Enschede (NL)

(72) Inventors: Tom Kamperman, Enschede (NL); Sara Tahan Latibari, Enschede (NL); Jenny Evelin Parraga Meneses, Enschede (NL)

(21) Appl. No.: 18/019,512

(22) PCT Filed: Aug. 3, 2021
(86) PCT No.: PCT/IB2021/057097

§ 371 Date: Feb. 3, 2023

(30) Foreign Application Priority Data
Aug. 3, 2020 (NL) 2026204

(51) Int. Cl. A61K 9/50; A61K 9/107; A61K 31/375; A61K 8/11; A61K 8/92; A61K 8/67; A61K 47/44; A23L 3/00; A61K 8/02

(52) U.S. Cl. CPC A61K 9/5036; A61K 9/107; A61K 31/375; A61K 8/11; A61K 8/92; A61K 8/67; A61K 47/44; A23L 3/00; A61K 8/025

(57) ABSTRACT

A reactive active compound is brought in a liquid. Said liquid, containing said active compound, is encapsulated by a surrounding shell layer and comprises an emulsion of a first liquid in a second liquid. Said active compound is dispersed in said first liquid. Said first liquid, containing said active compound, is dispersed in a second liquid that is substantially immiscible with said first liquid to form said emulsion. Said emulsion is encapsulated by said shell layer to preserve said reactive active compound in one or more a capsules that may be applied in a formulation.

The present invention relates to a method of preservation of a reactive active compound, wherein said active compound is brought in a liquid and said liquid, containing said active compound, is encapsulated by a surrounding shell layer. The invention moreover relates to such a capsule, comprising a core encapsulated in a shell layer, wherein said core comprises a liquid containing an active compound, as well as to a formulation, comprising a fluid material like a cream, lotion, gel, serum, cleanser, soap, shampoo, oil or clay that comprises a cosmetic, pharmaceutical, nutritious or organoleptic active compound.

In many applications and markets, active compounds are (micro)encapsulated to enhance shelf life, prevent degradation, increase solubility, or mask the taste of the active ingredient. In pharmaceutics, for instance, (micro)encapsulation strategies can be used to increase the solubility and to control the delivery of active pharmaceutical ingredients in the body. For cosmetics (micro)encapsulation strategies can be used to protect active cosmetic ingredients against oxygen-, temperature-, moist-, or light-induced degradation. In food (micro)capsules are used to increase the shelf-life, or mask the flavour of bad-tasting nutrients. Also (micro)encapsulation strategies can be used for the controlled release and/or prolonged prevalence of aroma's and ethereal oils by counteracting fast evaporation of fragrances.

Soft and hard shell capsules are widely used within the pharmaceutical, health and food industry and have gained an acceptance as they present pharmaceutical, cosmetic and health products in a form that is readily administered, consumed, applied and/or digested by a user. These capsules are generally made up of a shell and an active core material. The shell may be formed of readily digested materials, for example a soft gelatin capsule may comprise a mixture of gelatin, glycerol and water. Hard shell capsules generally comprise gelatin and water. Generally soft and hard shell capsules are suitable for encapsulating a wide range of pharmaceutical and health products in the form of a suspension.

Ascorbic acid (also known as vitamin C) is one of the most used natural reducing agents or antioxidants. This vitamin is well-known for its general essential protective role in health. It is a naturally organic compound which also plays a significant biological role in anti-oxidation, anti-aging, anti-cancer, and immune regulation. Therefore, ascorbic acid is added to many products, for instance inter alia in food, pharmaceutical, cosmetics, and dermatologic substances. In dermatology, vitamin C is known for its association with collagen synthesis as well as for its anti-oxidative nature, which ultimately reinforces the skin appearance by evoking the appearance of fine lines or wrinkles in the skin, as well as skin whitening effect, restoring skin elasticity, and eliminating harmful oxygen generated by ultraviolet light.

Ascorbic acid is easily degraded by oxidation, especially in aqueous solutions. Oxidation of ascorbic acid results in the formation of de-hydro-ascorbic acid, which is hypothesized to degrade into 2,3-diketogulonic acid. These reaction products have significantly less anti-oxidative properties and are characterized by a yellow to brown colour. Consequently, many vitamin C-laden products in, for example topical cosmetic creams, are characterized by a yellow to yellow/brown colouration, which indeed indicates that the vitamin C is, at least partially, oxidized and/or degraded and thus provides sub-optimal anti-oxidative properties.

Ascorbic acid degradation via oxidation or via other mechanisms is induced and affected by several physical and chemical factors such as elevated temperature, (UV) light, moisture, pH value, and the presence of (dissolved) oxygen. Due to this large variety of degradation factors, formulating stable formulations of ascorbic acid is not trivial. Several known stabilization or preservation methods have focused on the dehydration or chemical modification of ascorbic acid. Dried forms of ascorbic acid, however, are limited to application in equally dry formulations, such as tablets and powders. Many chemically modified forms of ascorbic acid that are intended to avoid degradation in moist environments have unnatural chemical structures that are biologically less active and therefore provide reduced health benefits.

Numerous alternative attempts have been made to conquer these limitations and find more stable compositions comprising ascorbic acid, for example by dissolving an appropriate derivative in an oil environment. To that end, water soluble vitamins such as the B group vitamins and ascorbic acid are generally presented in the form of a suspension in edible oil and encapsulated in a soft gelatin or hard shell capsule. Oils such as Soy Bean Oil are generally used. This hydrophobic environment can effectively minimize the oxidation of particularly vitamin C by reducing the amounts of water and oxygen to which ascorbic acid is exposed.

The vitamins may be used on their own as the active ingredient, or in combination with herbal materials such as Bioflavanoids, Rutin or with other vitamins. Ascorbic acid, for example, may be combined with other vitamins such as B groups, betacarotene, vitamin D and vitamin E or with minerals such as trace elements of iron, calcium, magnesium and zinc. Soft gelatin capsules containing vitamins such as ascorbic acid may be used for a number of therapeutic and complementary medicine purposes, for example as a component in an anti-oxidant therapy in conjunction with Betacarotene and Vitamin E.

Ascorbic Acid and other water-soluble vitamins, such as the B group vitamins, however have a certain solubility in the gelatin shell and can migrate from the core material to the shell if not completely insolubilised. Over time, the water soluble vitamin may enter or even cross the shell and may oxidise or react. In order to counteract this mechanism, international patent application WO 99/24021 discloses a method of immobilizing an encapsulated vitamin composition in that water soluble vitamin particles are coated with a material that is substantially insoluble in both the oily core material as well as in the shell of the capsule. The coated water soluble vitamin particles are of a size that are suitable for encapsulation in the form of an oily suspension that provides the core of the capsule.

Albeit being soluble in oil or fat, the solubility of these ascorbic acid derivatives and other active compounds in a lipid environment generally is relatively poor as compared to their solubility in water. Moreover, the fat-soluble derivatives of ascorbic acid appear to have practical limitations, for example, due to their rapid decomposition, poor solubility in aqueous formulations, and poor biological function and bio-activity. Coating particles of the active compound as thought by WO 99/24021 not only immobilizes the active compound but moreover deactivates the active compound. The coating needs to be removed to reactivate the active compound for instance by chemical reaction in the gastrointestinal tract of the user.

It is therefor inter alia an object of the invention to provide an improved method of preservation of a reactive active compound, particularly a water soluble active compound, as well as a capsule and formulation containing such a compound while maintaining the active compound in an active condition.

In order to attain said aim, a method of the type described in the opening paragraph is according to the invention characterized in that said active compound is brought in a first liquid, in that said first liquid, containing said active compound, is brought in a second liquid that is substantially immiscible with said first liquid, that an emulsion is formed containing said first liquid and said second liquid, and in that said emulsion is encapsulated by a solid shell layer, wherein both said first liquid and said shell layer are hydrophilic and said second liquid is hydrophobic.

A capsule of the type described in the opening paragraph is according to the invention characterized in that said liquid comprises an emulsion of a first liquid and a second liquid, said first and second liquid being substantially immiscible with one another and said first liquid comprising said active compound, wherein said core that comprises said emulsion is surrounded by a solid shell layer, wherein said first liquid is hydrophilic, wherein said second liquid is hydrophobic, and wherein said shell layer is hydrophilic. According to the invention, a formulation of the type as described in the opening paragraph is characterized in that said fluid material comprises a plurality of capsules containing an emulsion comprising a hydrophilic first liquid and a hydrophobic second liquid that are immiscible with one another, containing said active compound in said first liquid, confined within a hydrophilic solid shell layer. Said fluid particularly is a cosmetic base like a cream, lotion, gel, serum, cleanser, soap, shampoo, oil or clay that comprises a cosmetic, pharmaceutical, nutritious or organoleptic active compound within one or more capsules according to the invention.

Accordingly, the active compound may readily be dissolved or otherwise brought (e.g. suspended or dispersed) in a first liquid and then emulsified with a second liquid that will then serve as a barrier between the small, dispersed, particularly colloid, droplets of said first liquid, containing the active compound, and the surrounding shell. This protective environment has proven to protect said active compound against degradation and counteracts migration of said active compound to and across the shell layer. Leakage of the active compound through the shell layer to an unprotected or less protected (oxidizing) environment that would other wise lead to premature degradation may thereby be avoided or at least counteracted.

A particular embodiment of the method or capsule according to the invention is characterized in that said first liquid comprises water. An aqueous liquid is non-toxic and in many cases very practical to process particularly when used in combination with a water soluble active compound. Alternatively said first liquid may comprise a water-free solvent, particularly glycerine, glycerol or poly(ethylene glycol).

Similarly, a particular embodiment of the method or capsule according to the invention is characterized in that said second liquid comprises at least one of organic (vegetable or animal) oils, waxes and fats, and particularly in that said second liquid comprises an oil selected from a group consisting of essential oils, ethereal oils, macerated oils, triglyceride and mixtures or derivatives thereof and particularly comprises sunflower oil. Like water, also many of these naturally occurring oils and waxes are non-toxic and user-friendly. The hydrophobic environment created by such second liquid forms an effective barrier between the hydrophilic first liquid and the hydrophilic shell layer to prevent the first liquid, containing the active compound, from leaking through the shell layer.

A particularly favourable stability is achieved in a preferred embodiment of the method or capsule of the invention that is characterized in that said second liquid comprises a fat or wax in a form of micro-particles, preferably having a size smaller than 1 millimetre. These fat or wax micro-particles are believed to further immobilize hydrophilic droplets of the first liquid, containing the active compound, within the second liquid. In general fats and waxes are solid or creamy (malleable) at room temperature. Advantage may be taken of this natural nature of waxes and compounds to further immobilize the active compound within the capsule. Accordingly, a special embodiment of the method or capsule according to the invention is characterized in that said second liquid is a fat or wax that is processed in a liquid condition to solidify or have solidified between room temperature and at least approximately human body temperature. These waxes or fats may be processed in a liquid form to be able to create capsules, for instance by using the method of a co-pending European patent application by the same applicant that published as EP 3.436.188 A1 whose subject matter is herewith incorporated by reference. Subsequently the product may be stored at room temperature or below room temperature while the fat or wax contained in the capsule is in a solid state thereby counteracting migration of both the active compound out of the capsule as well as of environmental compounds into the capsule.

Applied in cosmetics or for pharmaceutical in or on the human body at body temperature, the wax will start to liquefy, having for instance a melting point just below 40° C., thereby mobilizing and subsequently releasing its active ingredient. A particular embodiment of the method and the capsule according to the invention are, hence, characterized in this respect in that said second liquid comprises a fat or wax that is processed in a liquid condition to solidify below 40° C., particularly between room temperature and human body temperature, in the second liquid.

Preferably said liquid condition is attained without adversely affecting the condition of the (aqueous) first fluid active compound. To that end, a further embodiment of the method or capsule according to the invention has the feature that said fat or wax has a melting point below about 90° C. This avoids boiling and consequently disintegration of aqueous first fluid droplets that are also dispersed throughout the second fluid and moreover suppresses thermally induced degradation of the active compound. Suitable candidates for these wax or fat micro-particles is for instance a wax that is selected from a group of montan wax, carnauba wax, glycol montanate, paraffin wax, candililla wax, beeswax, microcrystalline wax and ozocerite wax.

Particularly if applied in cosmetics and nutrients, ethereal, macerated and/or essential oils or waxes furthermore add favourable, pleasant organoleptic properties to the product. Examples of suitable organic lipophilic compounds are for instance:
vegetable oils, such as sunflower oil, corn oil, castor oil, palm oil, coconut oil, avocado oil, sweet almond oil, calophylum oil, lanolin, sesame oil, olive oil, jojoba oil, soybean oil, cottonseed oil, rapeseed oil, peanut oil, flaxseed oil, borage oil and vegetable oil derivatives,
essential oils, such as immortelle, lavender, german chamomile, neroli, peppermint oil, rosemary, rose oil, tea tree oil, dwarf pine, juniperberry, roast chestnut extract, birch leaf extract, hayseed extract, ethyl acetate, camphor, menthol, rosemary extract, eucalyptus oil,
macerated oils,
fatty acids, such as stearic acid, palmitic acid, behenic acid, myristic acid, lauric acid and capric acid,
fatty acid derivatives, such as fatty acid esters with short chain alcohols, such as isopropyl myristate, isopropyl palmitate and isopropyl stearate and dibutyl adipate;
medium and long chain fatty acids and their esters with polyols such as propylene glycol);
animal oils, such as tallow fat and marine oils, such as fish oils and seaweed oils or mixtures thereof;
nut oils, seed oils, waxes, such as paraffin wax, carnauba wax, candililla wax, beeswax, microcrystalline wax, ozocerite wax; and
triglyceride.

Preferably the capsule is loaded with a sufficient amount of the active compound. To that end a preferred embodiment of the method and capsule according to the invention is characterized in that said active compound is dissolved in said first liquid. According to this embodiment the active compound and first liquid are adapted to one another in that the active compound will be readily dissolvable in said liquid. Particularly high amounts of active compound may be loaded in the first liquid without substantially influencing the viscosity of the fluid concerned and its process behaviour.

In a further particular embodiment the method and capsule are thereby characterized in that said first liquid contains said active compound in a supersaturated condition. Super saturation is a solution that contains more of the dissolved active compound than could be dissolved by the solvent under normal circumstances. This allows for a further increase in the load of active compound in a capsule. Raising the temperature, pressure or volume of the liquid, may allow more compound to be dissolved than possible at normal, lower values. By changing back these parameters to their normal, lower values at a faster rate than required for the compound to precipitate or crystalize, the solution may become in a supersaturated condition, containing an increased amount of the active compound.

Particularly, the first liquid is a hydrophilic solution or suspension of said active compound, particularly an aqueous solution. The hydrophilic first liquid may, however, in itself already be suspended in a hydrophobic third liquid that in turn is suspended in a hydrophilic fourth liquid, and so on, to form a multiple-phase emulsion in the second liquid that may contain multiple active compounds or ingredients in either a hydrophilic or hydrophobic phase of such multiple emulsion. The hydrophobic second liquid in all these cases forms a continuous phase that surrounds dispersed kernels of hydrophilic fluid, ultimately containing the active compound. This hydrophobic continuous phase forms a barrier between the hydrophilic fluid and the hydrophilic shell layer, to thereby shield and confine the dispersed phase formed by the hydrophilic liquid together with the containing active compound within the protecting surrounding kernel.

In a specific embodiment, the method or capsule according to the invention is characterized in that said shell layer comprises a solid polymer network, particularly an interpenetrating network of two or more cross-linked polymers, and more particularly in that said polymer network comprises a hydrophilic polymer network, particularly comprising one or more poly-electrolytes or polysaccharides selected from agar, aliginate, chitosan, dextran, poly(ethylene glycol), collagen, gelatin, hyaluronic acid, carrageenan, fibroin, fibronectin, poly-L-Lysine (PLL), cellulose, graphene, polyethylenimine (PEI), poly(amidoamine) (PAA), dextran sulfate, silk, silk fibroin, Pectin, K-carrageenan, Iota carrageenan, Gellan gum, Guar gum, Tragacanth gum, Xanthan gum, Acacia gum, Karaya gum, Gelatin, Agar or Sodium carboxymethyl cellulose (S-CMC) all of these as naturally derived materials and/or synthetically derived materials including recombinant proteins and/or derivatives of these materials.

Particularly successful results were obtained in this respect with a further specific embodiment of the method and capsule, wherein said polymer network comprises a cross-linked or inter-penetrating aliginate network, particularly a calcium cross-linked aliginate network. The network may be further strengthened by incorporation of micro-particles and/or poly-electrolytes.

A preferred embodiment of the method and capsule according to the invention is characterized in that bivalent cations are added to said first liquid, particularly by means of an electrolyte supplying magnesium ions and/or calcium ions, more particularly a calcium or magnesium salt solution providing a ionic calcium concentration of at least 0,001 M in said first liquid, preferably at least 0.01 M, more particularly between 0.1 M and 1.0 M, even more particularly between 0.1 M and 0.5 M. This supply of divalent cations, particularly divalent magnesium and/or calcium ions, happens to stabilize and strengthen the calcium-aliginate network by preventing exhaustion of the cross linking calcium therein.

In general the present invention is based on the recognition that a reactive or otherwise vulnerable active compound that is prone to degradation, may effectively be preserved by bringing said compound in droplets of a first liquid and surrounding these droplets by a shielding second liquid and that such a system may be provided by a suitable emulsion of both liquids, said second liquid forming the continuous phase of said emulsion. This system can be confined to a solid capsule of desired size by encapsulated an appropriate volume of said emulsion by a suitable solid shell layer. As both liquids of the emulsion are in principle immiscible mutually, it is important to avoid phase separation and coalescence of the first liquid in the capsule. To that end, suitable surfactants, stabilizers and/or emulsifiers may be added to the system to counteract such separation. Such a surfactant may be:
a nonionic surfactant, such as polyoxyethylene sorbitan monolaureate (TWEEN®), (i.e., TWEEN®-20), polyoxyethylene 23 lauryl ether (BRIJ®-35) or polyoxyethylated octyl phenol (TRITON®);
a zwitterionic surfactant, such as 3-((3-cholamidopropyl) dimethylammonio)-1-propane sulfonate (CHAPS®);
a cationic surfactant; or
an anionic surfactant, such as cholate, deoxycholate, sodium dodecylsulfate, or TWEEN®-80.

As far as the method and capsule of the present invention are concerned, a particularly stable emulsion is created in a further preferred embodiment of the method and capsule according to the invention that are characterized in that said emulsion is stabilized by solid particles which adsorb onto an interface between said two liquids, and more particularly in that said solid particles are micro-particles or nano-particles that act as Pickering stabilizer.

These solid particles may be inorganic or organic. A particular embodiment of the method and capsule according to the invention are characterized in that said solid particles comprise micro-particles and/or nano particles that are selected from a group of particles containing silver, gold, fullerene, silicon, aluminum, calcium carbonate, zinc, mica, titanium, copper, platinum, silicic acid, lithium, magnesium, iron, magnetic nanoparticles, nanotubes, protein, cellulose, nano clay, including any of the oxidized forms of these materials such as silicon dioxide or silica.

Examples of inorganic nano or micro particles that may act as a Pickering stabilizer are:
Silica's, particularly fumed silica,
Metal oxides, like Fe₃O₄ nanoparticles, TiO₂, CuO,
Clays, particularly montmorillonite (MMT) and Laponites RD,
Layered double hydroxide (LDH), and
Carbons, like carbon nanotube (CNT), graphene oxide (GO), carbon black (CB).

Examples of organic nano or micro particles that may act as a Pickering stabilizer are:
Proteins, like bovine serum albumin coupled with PNIPAM,
Polysaccharide nanocrystals, like cellulose nanocrystals,
Chitin nanocrystals,
Starch nanocrystals,
Polymer particles, like poly(divinylbenzene-methacrylic acid) (P(DVB-MAA)) particles, Polystyrene (PS) and poly(methyl methacrylate) (PMMA) nanoparticles,
Hydroxyapatite, and
Chitosan.

The Pickering stabilization may be further enhanced by an appropriate pre-treatment. In that respect a further preferred embodiment of the method and capsule according to the invention are characterized in that said solid particles are hydrophobized, particularly by functionalizing or coating with dimethyldichlorosilane or poly(dimethylsiloxane), and more particularly in that solid particles comprise fumed silica nano-particles, preferably those that are post-treated with dimethyldichlorosilane (Si(CH₃)₂Cl₂).

To further enhance the Pickering stabilization of the emulsion, in a further embodiment of the method and capsule of the invention electrostatically charged particles or polymers are added to the first fluid having a polarity like that of the Pickering particles being used. Specifically a polyanioic polymer or negatively charged glycosaminoglycans may be added to stabilize a Pickering emulsion based on negatively charged Pickering particles, like fumed silica nano-particles. Conversely positively charged electrostatically charged particles, like chitosan, may be added to the first liquid to stabilize a Pickering emulsion that uses a positively charged Pickering agent. Particularly, said solid particles maybe charged negatively and said agent then may comprise a polyanionic polymer or a negatively charged glycosaminoglycan, more particularly hyaluronic acid, carageenan or acacia gum.

In view of use of the capsules for administering or other human or animal use, a further preferred embodiment of the method and capsule according to the invention are characterized in that said solid particles comprise micro-particles and/or nano particles that are bio-compatible and particularly biodegradable. As an example, said solid particle may comprise said solid particles comprise micro-particles and/or nano particles that are food grade stabilizers such as particles containing aliginate, starch, gelatin, fatty acids and/or derivatives thereof. These particles may be further enhanced in that said solid particles are hydrophobized by functionalizing or coating with a biodegradable compound, particularly with a compound from a group of amino acids, polyhydroxystearic acid, stearoyl glutamic acid, natural olive esters, jojoba ester, magnesium myristate, hydrogenated lecithin, lecithin, silica, isopropyl titanium triisostearate, dimethicone and methicone.

For stabilizing the active compound and, hence, improving shelf-life, it may further be favourable to tweak and tune its chemical environment of particularly the first liquid. To that end, a further particular embodiment of the method and capsule according to the invention are characterized in that an acid, a base and/or a buffer agent is added to said first liquid, maintaining a pH of said first liquid at a predetermined level that further aids in stabilizing said active compound, particularly ascorbic acid, hyaluronic acid, a zwitterionic sulfonic acid buffering agent, more particularly 4-(2-hydroxyethyl)-1-piperazineethanesulfonic acid (HEPES), or an acid, more particularly metaphosphoric acid, citric acid, perchloric acid, acetic acid or orthophosphoric acid. Said acidity (pH value) may, specifically, be held at a value of below pH=6-7 to promote an acidic condition of the first liquid.

A particularly stable emulsion is reached in a further preferred embodiment of the method and capsule according to the invention that are characterized in that ascorbic acid is added to said first liquid in a form of a micronized ascorbic acid powder, having a maximum particle size below 150 micron, particularly at a concentration of more than 25 wt %.

In principle the capsules according to the invention can be realised in a wide range of dimensions. The application of the active compound has, however, been found particularly favourable if provided in the form of micro or nano capsules, having sub-micron to millimetre size. Accordingly a further preferred embodiment of the method according the invention is characterized in that said shell layer encloses a volume of less than one millilitre thereby forming a micro-capsule containing said emulsion comprising said first liquid, said second liquid and said active compound in said first liquid. Similarly a further preferred embodiment of the capsule according the invention is characterized by a Feret diameter averaged over all directions of between 1 μm and 10 mm, particularly between 10 μm and 10 mm, particularly between 50 micron and 5 mm. In this respect capsules that are intended to remain visible are preferably created having a dimeter of between 500 micron and 5000 micron, whereas smaller micro-capsules may be created having a size between 50 micron and 500 micron.

Particularly, a shell layer thickness constitutes less than 25% of the diameter of said capsule and the core comprises said first liquid in a ratio of at least 1:100, particularly at least 1:10, more particularly at least 1:3, compared to said second liquid. Particularly, the first liquid forms a dispersed phase in a continuous phase that is formed by said second liquid and as such will not exceed the content of said second liquid. Said active compound constitutes preferably between 10% and 50% by weight of said first liquid.

In principle a wide variety of active ingredients may be processed with the method according to the invention and supplied in the form of one or more capsules according to the invention. However, the present invention is particularly useful for active compounds that would be unstable or prone to rapid degradation when exposed to (excessive) moist or air. The invention proves especially beneficial in certain specific embodiments of the method and capsule according to the invention that are characterized in that said active compound comprises at least one active compound that is selected from a group of pharmaceutical agents, flagrances, cosmetic agents, flavours and nutrients, wherein more particularly said active compound comprises at least one active compound that is selected from a group of vitamins, anti-oxidants, proteins and/or derivatives thereof.

As an example, the method and capsule according to the invention may be used in embodiments, wherein said active compound comprises at least one vitamin, particularly a vitamin that is selected from a group containing thiamine, riboflavin, nicotinic acid, pantothenic acid, pyridoxine, biotin, folic acid, cyanocobalamin, lipoic acid, ascorbic acid, lecithin, glycyrrhizin acid, retinol, retinol palmitate, tocopherol, tocopherol acetate, salicylic acid, benzoyl peroxide, and azelaic acid and/or derivatives thereof, more particularly ascorbic acid and/or derivatives thereof.

As another example, the method and capsule according to the invention may be used in embodiments, wherein said active compound comprises at least one anti-oxidant, particularly an anti-oxidant that is selected from a group containing poly-phenols, thiol-based components, sulphite and derivatives thereof. These active compounds may be used, for instance, as nutritious supplements or for pharmaceutical treatment, in which case they are likely to be administered orally. The shell layer may be formulated to survive the acidic environment of the human stomach to be digested in the more downstream portion of gastrointestinal tract of the user to release its contents. Specifically pro- and prebiotics may be administered particularly effectively in this manner. Particularly, said shell layer may carry a coating, preferably an edible coating, particularly a hydrophobic coating containing nano-particles or a wax like carnauba wax.

The capsules may also be used for cosmetic purposes, for instance for skin therapy or skin protection. To that end a special embodiment of the capsule according to the invention is characterized in that said shell layer is configured to break or rupture under a mechanical load. The configuration of the shell layer in this case promotes the release of its active contents when being applied, particularly rubbed out, onto the skin. The shell layer may be provided with a colouring, particularly coloured particles, more particularly water-insoluble pigments, even more particularly UV-absorbing or UV-reflecting pigments.

Hereinafter the invention will be described in further detail with reference to a number of explanatory embodiments and an accompanying drawing. In the drawing:

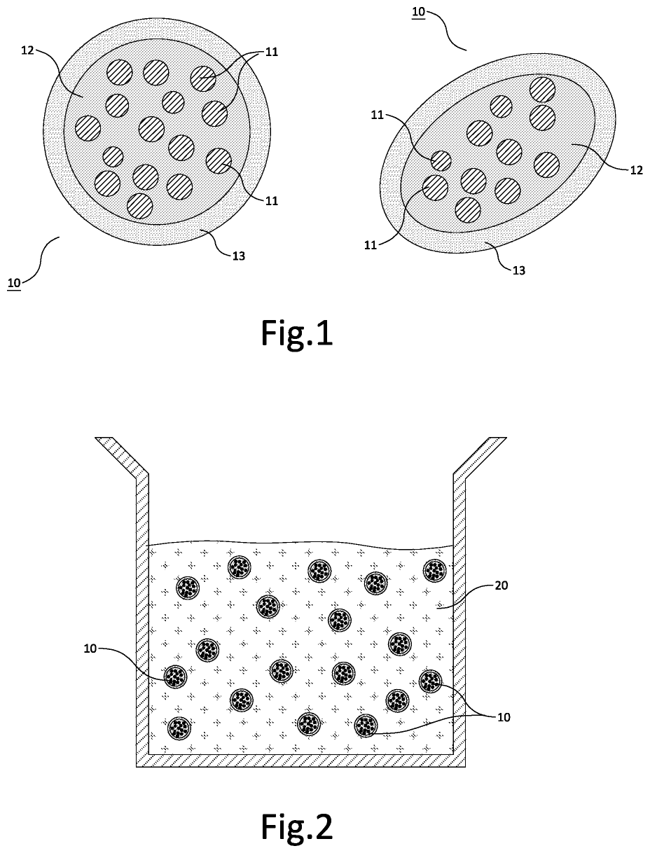

FIG. 1 shows a specific example of a capsule according to the invention; and

FIG. 2 shows an embodiment of a formulation according to the invention.

It is noted that the figures are drawn purely schematically and not necessarily to a same scale. In particular, certain dimensions may have been exaggerated to a more or lesser extent to aid the clarity of any features. Similar parts are generally indicated by a same reference numeral throughout the figures.

The capsules 10 that are shown in FIG. 1 are manufactured using a method according to the invention and resemble the protective nature that is provided by the invention to a vulnerable active compound that would otherwise be prone to oxidation or other degradation when exposed to ambient air, for instance. The active compound comprises for example ascorbic acid, being readily soluble in water. By dissolving micronized powder, for instance having a modal particle size of below around 75-100 micron a large quantity of for instance between 20 and 25 wt % ascorbic acid may be brought in an aqueous hydrophilic phase 11. The hydrophilic phase 1 an emulsion with a hydrophobic continuous phase 12, for instance containing a vegetable oil like sunflower oil. Solid nano-clay particles of laponite clay may be added to the mixture to act as a Pickering stabilizer to stabilize the emulsion, counteracting coalescence of the aqueous phase droplets 11 and phase separation. The emulsion comprised of small dispersed hydrophilic droplets 11 surrounded by a hydrophobic continuous phase 12 is encapsulated within a hydrophilic solid shell layer 13, for instance created by a solidified cross-linked hydrophilic calcium-aliginate network. The capsules 10 typically enclose a volume of less than one millilitre thereby forming a micro-capsule containing said emulsion comprising said first liquid 11 and said second liquid 12, protecting said active compound in said first liquid.

The formulation of FIG. 2 comprises a plurality of micro-capsules 10 as shown in FIG. 1 that contain an appropriate active compound. The capsules are distributed is a suitable fluid material 20 like a cream, lotion, gel, serum, cleanser, soap, shampoo, oil or clay to provide a cosmetic, pharmaceutical, nutritious or organoleptic product that expresses the desired properties of the active compound.

### Example 1: Preservation of Ascorbic Acid (AA) Via Encapsulation in Oil-Filled Calcium-Aliginate Capsules

AA is encapsulated in oil-filled aliginate capsules. Specifically:
(i) creation of a preserved AA laden sample.

50% (w/v) AAsodium salt (sodium L-ascorbate) is dissolved in water, which is then emulsified in sunflower oil. Stable surfactant-free water-in-oil (w/o) emulsion is generated by shaking and ultrasonically treating degassed water and oil solutions.

The AA-laden w/o emulsion is kept at 70° C. while it is jetted with a flow rate of 13 ml/min from the centre nozzle of a coaxial nozzle assembly (OD=1.6 mm and ID=0.41 mm) as disclosed in the aforementioned co-pending European patent application by applicant, that published as EP3.436.188 A1, whose subject matter is incorporated by reference.

Concurrently a 0.5% (w/v) sodium aliginate in water solution (WAKO, 1%^{˜}80-120 cP) of room temperature is jetted from the outer nozzle of the same coaxial nozzle assembly at a flow rate of 55 ml/min, resulting in a compound jet consisting of AA-laden w/o emulsion encapsulated by a sodium aliginate solution. The coaxial nozzle is modulated, using a vibrating element at a frequency of approximately 150 Hz which causes the controlled breakup of the compound jet into a stream of substantially mono-disperse, i.e. substantially uniformly sized, compound droplets with typically a coefficient of variation in size or diameter of less than 10%.

Via a so called ‘in-air microfluidics’ method, as described in the aforementioned co-pending application (EP 3.436.188), the droplet stream was combined with an intact, i.e. uninterrupted jet of 0.2 M calcium in water solution, resulting in the formation of compound hydrogel capsules consisting of a calcium-aliginate shell layer filled with a core of AA-laden w/o emulsion. These hydrogel capsules were stored in water and incubated for 6 weeks at 40° C.
ii creation of a non-preserved AA laden reference sample

For comparison purposes a non-preserved AA control sample was created by dissolving 0.5% (w/v) AA in water, i.e. having a same final concentration as the preserved sample. Also this non-preserved AA in water solution was incubated for 6 weeks at 40° C.

Comparing a colour change of the preserved sample to that of the similarly incubated (6 weeks at 40° C.) reference solution showed a brown colouring of the reference solution, that is typical for the oxidation product of AA, while the preserved sample showed no significant colouring. This reveals that the encapsulation of AA in oil-filled capsules, according to the invention, reduces colouring, indicating suppression or even prevention of AA degradation.

### Example 2: Both Physical Encapsulation and the Presence of Calcium

To investigate the mechanism of AA preservation via encapsulation in calcium-aliginate shells, three conditions were compared:
(i) AA preserved as described in experiment 1;
(ii) Non-preserved control: same as (i), but using a 0.2M sodium chloride solution instead of a 0.2M calcium chloride solution, which will prevent the formation of gelled calcium-aliginate capsules and thus the encapsulation of the AA-laden w/o emulsion;
(iii) Non-preserved control: same as (i), but the 0.2M calcium chloride jet was not impacted with the compound droplets prior to collection in the collector bath, which prevents the in-flight formation of stable calcium-aliginate capsules and thus the encapsulation of the AA-laden w/o emulsion.

The concentration of AA in all conditions was at least 5% (w/v) in the w/o emulsions and at least 0.5% (w/v) in the final sample volumes. Comparison of the colour change after incubation at 40° C. for 4 weeks showed no significant colouring of the preserved sample (1), comprising AA-laden hydrogel capsules having a calcium cross-linked aliginate shell layer, while sample (ii) showed significant browning after 4 weeks and sample (iii) showed slight colouring. This revealed that the presence of divalent calcium ions has a preserving effect on AA.

### Example 3: Oil-Filled Capsules Using Pickering Emulsifiers

Fumed silica nanoparticles post-treated with dimethyl-dichlorosilane (DDS) (Evonik, Aerosil R972) were added to the core of the capsules by dispersing 2% (w/v) of nano-particles in the oil phase. The oil that contains hydrophobized silica particles is then processed following the same method as described in example 1, except that a supersaturated AA-solution containing 100% (w/v) was used. In this example calcium carbonate (CaCO₃) is added to the dispersed AA-lade phase before emulsification to load the capsules with excess CaCO₃ that will aid in maintaining a stable the calcium-aliginate shell over time.

This creates AA-laden silica-oil filled calcium-aliginate capsules, having a final AA concentration in the w/o emulsion (i.e. the core compound) of at least 10% (w/v), a final CaCO₃ concentration in the w/o emulsion of 2% (w/v) and a final hydrophobized silica concentration in the w/o emulsion of 2% (w/v). The capsules were washed two times with demineralised water and incubated in demineralised water at 40° C. for 23 days.

After 23 days the samples showed no significant colouring, revealing an effective preservation of both the AA active compound in the dispersed phase as well as of the w/o emulsion itself and the surrounding calcium-aliginate shell layer.

### Example 4: Oil-Filled Capsules Using Pickering Emulsifiers in Core and Additives in Shell

Fumed silica nano-particles are post-treated with dimethyl-dichlorosilane (DDS) (Evonik, Aerosil R972) and added to the core of the capsules by dispersing 4% (w/v) of nano-particles in an oil phase. The oil that contains these hydrophobized silica particles and is processed following the same method as described in example 1 together with a aqueous solution of ascorbic acid to form an emulsion, except that a saturated L-ascorbic acid fine powder containing 40% (w/v) was used for the solution.

Further 0.5% laponite XL21XR nano-clay is added to the 0.5% aliginate phase in the shell during the encapsulation. This creates AA-laden silica-oil filled calcium-aliginate/laponite capsules, having a final AA concentration in the w/o emulsion (i.e. the core compound) of at least 10% (w/v), and a final hydrophobized silica concentration in the w/o emulsion of 4% (w/v).

The capsules are washed two times with demineralised water and incubated in demineralised water, 0.2M CaCl2+10 wt % ethanol solution, clear hand gel, shampoo, and body lotion at 40° C. for 4 weeks. After 4 weeks the samples showed no significant colouring compared to a similar sample containing the same emulsion without encapsulation and L-ascorbic acid bulk solution in water. This reveals an effective preservation of both the AA active compound in the dispersed phase as well as of the w/o emulsion itself by the surrounding calcium-aliginate shell layer.

### Example 5: Agar Reinforced Encapsulated Capsules

Similar capsules are prepared same as in example 4, but additionally 0.5 wt % agar solution was used for forming the shell composition. This results in ascorbic acid containing emulsion capsules within a 1% laponite XL21XR/0.5% aliginate/0.5% agar solid shell layer. These capsules were stored for 4 weeks in water, shampoo, body lotion and 0.2M CaCl2+10% ETOH to show hardly no colouring compared to the bare emulsion in the same base.

### Example 6: Layer-by-Layer Coating of the Solid Shell

Capsules are prepared similar to example 4, but with coating by a layer using electrostatically interaction of layer-by-layer method (LBL). The aliginate shell is coated by a positively charged biopolymer, such as chitosan, to enhance the barrier property and stability of L-ascorbic acid. Chitosan is applied as a polycationic polymer to ionically crosslink the aliginate shell. Initially, a stable 5 wt % chitosan stock solution is prepared in aid of 1% (v/v) hydrochloric acid (HCl) while stirring for several hours at 50° C. Afterwards, the 5 wt % chitosan solution is neutralized by adjusting the PH to about 6-7 while adding sodium hydroxide (NaOH) solution. Then the neutralized solution is diluted to make a 0.5 wt % chitosan solution to be used in the coating process.

Vitamin C encapsulated beads (capsules) in an aliginate shell are coated by a chitosan/aliginate bilayer. First the capsules are rinsed with water and then incubated in 0.5 wt % neutralized chitosan solution while stirring slowly for 15 min. The incubated beads are filtered and rinsed with water, then incubated and stirred slowly in 0.5 wt % of aliginate solution for another 15 min. To avoid the aggregation of capsules, slow movement (stirring) is applied while incubating the beads. To achieve a denser coating, a salt solution (NaCl solution) is applied to coat the same capsules by ionic crosslinking of chitosan/aliginate. The purpose of using salts is to ensure attaining a stable thickness by alternating deposition. 0.8M NaCl solution is applied in all incubation and rinsing steps.

### Example 7: Improved Stabilization Using Solidified Solid Wax Particles

Similar capsules are prepared same as in example 4, but instead of fumed silica nano-particles, 2 wt % micronized carnauba wax (Microcare 350, Micro Powder Inc.) is added to the oil phase (i.e., the second liquid). Alternatively, a sample is prepared containing an oil phase with 4 wt % hydrophobized fumed silica as well as 2 wt % micronized carnauba wax. The capsules are then incubated for 0.5, 1, 2, 5, 10, and 20 minutes at 40, 60, 75, or 90 degrees Celcius. 5 minutes incubation at 90 degrees appears optimal for melting the micronized wax particles in the capsules in order to render the emulsion more stable after the wax has again solidified in the oily phase.

The capsules were tested for ascorbic acid stability by incubating them in water, shampoo, body lotion and 0.2M CaCl₂+10% ETOH, at 40 degrees Celsius. After this treatment the capsules show hardly no yellow colouring as compared to the same particles that were mechanically disrupted at the start of the stability test, indicating an improved chemical stability (i.e., preservation) of the encapsulated ascorbic acid within capsules containing a Pickering and wax stabilized emulsion of the dispersed ascorbic acid aqueous phase.

### Example 8: Electrostatically Loading the First Liquid

An aqeuous phase from the below list was added to 6% wt Aerosil R972 to act as Pickering stabilizer in sunflower oil. Three homogenization cycles using T25 Ultra-Turrax with 525N-25F dispering element were applied to form a Pickering emulsion: 1 cycle of 2 min at 10.000 rpm and 2 cycles of 1 min at 10.000 rpm. In between the cycles the emulsion was mixed manually to ensure a total incorporation of the phases.
1. Water
2. Water+1 wt % NaCl
3. Water+HCl (pH 2)
4. Water+20 wt % hyaluronic acid
5. Water+20 wt % hyaluronic acid+1 wt % NaCl
6. Water+2 wt % carrageenan
7. Water+2 wt % acacia gum
8. Water+2 wt % acacia gum+1 wt % NaCl
9. Water+1 wt % chitosan

The resulting emulsion of samples 4-7 appeared to be significantly more stable as the other samples. This supports that the water-in-oil Pickering emulsion based on negatively charged Aerosil R972 as Pickering agent is further stabilized by the addition of polyanionic polymers or (negatively charged) glycosaminoglycans, notably by hyaluronic acid, carrageenan or acacia gum, to the aqueous phase.

Although the invention has been described herein before with reference to merely a limited number of explanatory embodiments, it should be understood that the invention is by no means limited to those examples. On the contrary many more variations and embodiments are feasible to a skilled person within the framework of the present invention without requiring him or her to exercise any inventive skill. Particularly, further components other than the first fluid may also contain one or more active compounds, notably also the hydrophobic second fluid. The first fluid may be emulsified directly with the second fluid or may find itself in an emulsion with one or more further fluids to be jointly emulsified or mixed with the second fluid. Also the second fluid may itself consist of an emulsion with a further fluid. Each fluid may be used as a carrier for one or more specific active compounds or ingredients.

1. A method of preservation of a reactive active compound, wherein said active compound is brought in a liquid and said liquid, containing said active compound, is encapsulated by a surrounding shell layer, wherein said active compound is brought in a first liquid, wherein said first liquid, containing said active compound, is brought in a second liquid that is substantially immiscible with said first liquid, that an emulsion is formed containing said first liquid and said second liquid, and wherein said emulsion is encapsulated by a solid shell layer, wherein both said first liquid and said shell layer are hydrophilic and said second liquid is hydrophobic.

2. The method according to claim 1, wherein said first liquid comprises water or a water-free hydrophilic solvent, particularly glycerine, glycerol or poly(ethylene glycol).

3. The method according to claim 1, wherein said second liquid comprises at least one of organic (vegetable or animal) oils, waxes and fats.

4. The method according to claim 3, wherein said second liquid comprises a fat or wax in a form of micro-particles, preferably having a size smaller than 1 millimetre.

5. The method according to claim 3, wherein said second liquid comprises a fat or wax that is processed in a liquid condition to solidify below 40° C., particularly between room temperature and human body temperature, in the second liquid.

6. The method according to claim 3, wherein said second liquid comprises a solid wax or fat that was added in the form of solid micro-particles and subjected to a heat treatment to at least partly melt said wax or fat micro-particles while being in the second liquid.

7. The method according to claim 3, wherein said fat or wax has a melting point below 90° C.

8. The method according to claim 7, wherein said fat or wax is selected from a group of montan wax, carnauba wax, glycol montanate, paraffin wax, candililla wax, beeswax, microcrystalline wax and ozocerite wax.

9. The method according to claim 3, wherein said second liquid comprises an oil selected from a group consisting of essential oils, ethereal oils, macerated oils, triglyceride and mixtures or derivatives thereof and particularly comprises sunflower oil.

10. The method according to claim 1, wherein said active compound is dissolved in said first liquid, wherein said first liquid contains said active compound preferably in a supersaturated condition.

11. The method according to claim 1, wherein said first liquid is a hydrophilic solution or suspension of said active compound, particularly an aqueous solution, while said second liquid is hydrophobic.

12. The method according to claim 1, wherein said shell layer comprises a polymer network, particularly an interpenetrating network of two or more cross-linked polymers.

13. The method according to claim 12, wherein said polymer network comprises a hydrophilic polymer network, particularly comprising one or more poly-electrolytes or polysaccharides selected from agar, aliginate, chitosan, dextran, poly(ethylene glycol), collagen, gelatin, hyaluronic acid, carrageenan, fibroin, fibronectin, poly-L-Lysine (PLL), cellulose, graphene, polyethylenimine (PEI), poly(amidoamine) (PAA), dextran sulfate, silk, silk fibroin, Pectin, K-carrageenan, Iota carrageenan, Gellan gum, Guar gum, Tragacanth gum, Xanthan gum, Acacia gum, Karaya gum, Gelatin, and Sodium carboxymethyl cellulose (S-CMC) all of these as naturally derived materials and/or synthetically derived materials including recombinant proteins and/or derivatives of these materials.

14. The method according to claim 13, wherein said polymer network comprises a cross-linked or inter-penetrating aliginate network, particularly a calcium cross-linked aliginate network.

15. The method according to claim 14, wherein bivalent cations are added to said first liquid, particularly by means of an electrolyte supplying magnesium ions and/or calcium ions, more particularly a calcium or magnesium salt solution providing an ionic calcium concentration of at least 0,001 M in said first liquid, preferably at least 0.01 M, more particularly between 0.1 M and 1.0 M, even more particularly between 0.1 M and 0.5 M.

16. The method according to claim 1, wherein said emulsion is stabilized by solid particles which adsorb onto an interface between said two liquids, particularly solid particles that are micro-particles or nano-particles that act as Pickering stabilizer.

17. The method according to claim 16, wherein said solid particles are charged particles and wherein a electrostatically charged agent having a same polarity as said solid particle is added to the first liquid, particularly said solid particles being charged negatively and said agent comprising a polyanionic polymer or a negatively charged glycosaminoglycan, more particularly hyaluronic acid, carageenan or acacia gum.

18. The method according to claim 16, wherein said solid particles comprise micro-particles and/or nano particles that are selected from a group of particles containing silver, gold, fullerene, silicon, aluminum, calcium carbonate, zinc, mica, titanium, copper, platinum, silicic acid, lithium, magnesium, iron, magnetic nanoparticles, nanotubes, protein, cellulose and clay, particularly laponite clay, including any of the oxidized forms of these materials such as silicon dioxide or silica.

19. The method according to claim 16, wherein said solid particles are hydrophobized, particularly by functionalizing or coating with chlorosilane or silanol, particularly (alkyl)chlorosilane, trimethylsilanol, dimethyldichlorosilane or poly(dimethylsiloxane).

20. The method according to claim 19, wherein said solid particles comprise fumed silica nano-particles, preferably post-treated with dimethyldichlorosilane (Si(CH₃)₂Cl₂).

21. The method according to claim 16, wherein said solid particles comprise micro-particles and/or nano particles that are bio-compatible and particularly biodegradable.

22. The method according to claim 21, wherein said solid particles comprises a biodegradable compound or are hydrophobized by functionalizing or coating with a biodegradable compound, particularly with a compound from a group of amino acids, polyhydroxystearic acid, stearoyl glutamic acid, natural olive esters, jojoba ester, magnesium myristate, hydrogenated lecithin, lecithin, silica, isopropyl titanium triisostearate, dimethicone and methicone.

23. The method according to claim 22, wherein said solid particles comprise micro-particles and/or nano particles that are food grade stabilizers such as particles containing aliginate, starch, gelatin, fatty acids and/or derivatives thereof.

24. The method according to claim 1, Wherein an acid, a base and/or a buffer agent is added to said first liquid, maintaining a pH of said first liquid at a predetermined level that further aids in stabilizing said active compound, particularly one or more of ascorbic acid, hyaluronic acid and a zwitterionic sulfonic acid buffering agent, more particularly 4-(2-hydroxyethyl)-1-piperazineethanesulfonic acid (HEPES), metaphosphoric acid, citric acid, perchloric acid, acetic acid or orthophosphoric acid.

25. The method according to claim 24, wherein ascorbic acid is added to said first liquid in a form of a micronized ascorbic acid powder, having a maximum particle size below 150 micron, particularly at a concentration of more than 25 wt %.

26. The method according to claim 1, wherein said shell layer encloses a volume of less than one millilitre thereby forming a micro-capsule containing said emulsion comprising said first liquid, said second liquid and said active compound in said first liquid.

27. The method according to claim 1, wherein said active compound comprises at least one active compound that is selected from a group of pharmaceutical agents, flagrances, cosmetic agents, flavours and nutrients.

28. The method according to claim 27, wherein said active compound comprises at least one active compound that is selected from a group of vitamins, anti-oxidants, proteins and/or derivatives thereof.

29. The method according to claim 28, wherein said emulsion comprises at least one vitamin, particularly a vitamin that is selected from a group containing thiamine, riboflavin, nicotinic acid, pantothenic acid, pyridoxine, biotin, folic acid, cyanocobalamin, lipoic acid, ascorbic acid, lecithin, glycyrhizin acid, retinol, retinol palmitate, tocopherol, tocopherol acetate, salicylic acid, benzoyl peroxide, and azelaic acid and/or derivatives thereof, more particularly ascorbic acid and/or derivatives thereof.

30. The method according to claim 28, wherein said active compound comprises at least one anti-oxidant, particularly an anti-oxidant that is selected from a group containing poly-phenols, thiol-based components, sulphite and derivatives thereof.

31. A capsule, comprising a core encapsulated in a shell layer, wherein said core comprises a liquid containing an active compound, wherein said liquid comprises an emulsion of a first liquid and a second liquid, said first and second liquid being substantially immiscible with one another and said first liquid comprising said active compound, wherein said core that comprises said emulsion is surrounded by a solid shell layer, wherein said first liquid is hydrophilic, wherein said second liquid is hydrophobic, and wherein said shell layer is hydrophilic.

32. The capsule according to claim 31, wherein said first liquid contains water or a water-free solvent, particularly glycerine, glycerol or poly(ethylene glycol).

33. The capsule according to claim 31, wherein said second liquid comprises at least one of organic (vegetable or animal) oils, waxes and fats.

34. The capsule according to claim 33, wherein said second liquid comprises a wax in a form of micro-particles, preferably having a size smaller than 1 millimetre.

35. The capsule according to claim 33, wherein said second liquid comprises a fat or wax that is processed in a liquid condition to solidify below 40° C., particularly between room temperature and human body temperature.

36. The capsule according to claim 33, wherein said fat or wax has a melting point below 90° C.

37. The capsule according to claim 36, wherein said fat or wax is selected from a group of montan wax, carnauba wax, glycol montanate, paraffin wax, candililla wax, beeswax, microcrystalline wax and ozocerite wax.

38. The capsule according to claim 33, wherein said second liquid is an organic oil that is selected from a group consisting of essential oils, ethereal oils, macerated oils, triglyceride and mixtures or derivatives thereof and particularly comprises sunflower oil.

39. The capsule according to claim 31, wherein said active compound is dissolved in said first liquid.

40. The capsule according to claim 39, wherein said first liquid contains said active compound in a supersaturated condition.

41. The capsule according to claim 31, characterized in said first liquid is a hydrophilic solution or suspension of said active compound, particularly an aqueous solution.

42. The capsule according to, wherein said shell layer comprises a polymer network, particularly an interpenetrating network of two or more cross-linked polymers.

43. The capsule according to claim 42, wherein said polymer network comprises a hydrophilic polymer network, particularly comprising one or more poly-electrolytes or polysaccharides selected from agar, aliginate, chitosan, dextran, poly(ethylene glycol), collagen, gelatin, hyaluronic acid, carrageenan, fibroin, fibronectin, poly-L-Lysine (PLL), cellulose, graphene, polyethylenimine (PEI), poly(amidoamine) (PAA), dextran sulfate, silk, silk fibroin, Pectin, K-carrageenan, Iota carrageenan, Gellan gum, Guar gum, Tragacanth gum, Xanthan gum, Acacia gum, Karaya gum, Gelatin, Agar or Sodium carboxymethyl cellulose (S-CMC) all of these as naturally derived materials and/or synthetically derived materials including recombinant proteins and/or derivatives of these materials, wherein said polymer network particularly comprises a hydrophilic calcium-aliginate network.

44. The capsule according to claim 43, wherein said first liquid comprises bivalent cations, particularly an electrolyte supplying magnesium ions and/or calcium ions, more particularly a calcium or magnesium salt solution providing a ionic calcium concentration of at least 0,001 M in said first liquid, preferably at least 0.01 M, more particularly between 0.1 M and 1.0 M, even more particularly between 0.1 M and 0.5 M.

45. The capsule according to claim 31, wherein said emulsion is stabilized by solid particles which adsorb onto an interface between said two liquids, wherein said solid particles are particularly micro-particles or nano-particles that act as Pickering stabilizer.

47. The capsule according to claim 45, wherein said solid particles are charged particles and wherein a electrostatically charged agent having a same polarity as said solid particle is added to the first liquid, particularly said solid particles being charged negatively and said agent comprising a polyanionic polymer or a negatively charged glycosaminoglycan, more particularly hyaluronic acid, carageenan or acacia gum.

47. The capsule according to claim 45, wherein said solid particles comprise micro-particles and/or nano particles that are selected from a group of particles containing silver, gold, fullerene, silicon, aluminum, calcium carbonate, zinc, mica, titanium, titanium, copper, platinum, silicic acid, lithium, magnesium, iron, magnetic nanoparticles, nanotubes, protein, cellulose, clay, particularly laponite clay, including any of the oxidized forms of these materials such as silicon dioxide or silica.

48. The capsule according to one or more of claim 48, wherein said solid particles are hydrophobized, particularly by functionalizing or coating with dimethyldichlorosilane or poly(dimethylsiloxane).

49. The capsule according to claim 48, wherein said solid particles comprise fumed silica nano-particles, preferably post-treated with dimethyldichlorosilane (Si(CH₃)₂Cl₂).

50. The capsule according to claim 45, wherein said solid particles comprise micro-particles and/or nano particles that are bio-compatible and particularly biodegradable.

51. The capsule according to claim 50, wherein said solid particles comprises a biodegradable compound or are hydrophobized by functionalizing or coating with a biodegradable compound, particularly a compound from a group of amino acids, polyhydroxystearic acid, stearoyl glutamic acid, natural olive esters, jojoba ester, magnesium myristate, hydrogenated lecithin, lecithin, silica, isopropyl titanium triisostearate, dimethicone and methicone.

52. The capsule according to claim 51, wherein said solid particles comprise micro-particles and/or nano particles that are food grade stabilizers such as particles containing aliginate, starch, gelatin, fatty acids and/or derivatives thereof.

53. The capsule according to claim 31, wherein said first liquid comprises an acid, a base and/or a buffer agent, maintaining a pH of said first liquid at a predetermined level that further aids in stabilizing said active compound, particularly one or more of ascorbic acid, hyaluronic acid and a zwitterionic sulfonic acid buffering agent, more particularly 4-(2-hydroxyethyl)-1-piperazineethanesulfonic acid (HEPES), metaphosphoric acid, citric acid, perchloric acid, acetic acid or orthophosphoric acid.

54. The capsule according to claim 31, wherein said active compound comprises at least one active compound that is selected from a group of pharmaceutical agents, cosmetic agents, fragrances, flavours and nutrients.

55. The capsule according to claim 54, wherein said active compound comprises at least one compound that is selected from a group of vitamins, anti-oxidants, proteins and/or derivatives thereof.

56. The capsule according to claim 54, wherein said emulsion comprises at least one vitamin, particularly a vitamin that is selected from a group containing thiamine, riboflavin, nicotinic acid, pantothenic acid, pyridoxine, biotin, folic acid, cyanocobalamin, lipoic acid, ascorbic acid, lecithin, glycyrhizin acid, retinol, retinol palmitate, tocopherol, tocopherol acetate, salicylic acid, benzoyl peroxide, and azelaic acid and/or derivatives thereof, more particularly ascorbic acid and/or derivatives thereof.

57. The capsule according to claim 55, wherein said active compound comprises at least one anti-oxidant, particularly an anti-oxidant that is selected from a group containing poly-phenols, thiol-based components, sulphite and derivatives thereof.

58. The capsule according to claim 31, wherein said shell layer carries a coating, preferably an edible coating, particularly a hydrophobic coating containing nano-particles or a wax like carnauba wax.

59. The capsule according to claim 31, wherein said shell layer is configured to break or rupture under a mechanical load, particularly a manually applied mechanical load, more particularly by squeezing or chewing.

60. The capsule according to claim 31, wherein said shell layer is provided with a colouring, particularly coloured particles, more particularly water-insoluble pigments, even more particularly UV-absorbing or UV-reflecting pigments.

61. The capsule according to claim 31, characterized by a substantially spherical shape.

62. The capsule according to claim 61, characterized by a Feret diameter averaged over all directions of between 1 μm and 10 mm, particularly between 10 μm and 10 mm, particularly between 50 micron and 5 mm.

63. The capsule according to claim 62, wherein a shell layer thickness constitutes less than 25% of the diameter of said capsule.

64. The capsule according to claim 63, wherein a ratio of said first liquid to said second liquid is at least 1:100, particularly at least 1:10, more particularly at least 1:3.

65. The capsule according to claim 63, wherein said active compound constitutes 10-50% by weight of said first liquid.

66. A formulation, comprising a fluid material like a cream, lotion, gel, serum, cleanser, soap, shampoo, oil or clay that comprises a cosmetic, pharmaceutical, nutritious or organoleptic active compound, wherein said fluid material comprises a plurality of capsules comprising a hydrophilic first liquid and a hydrophobic second liquid that are immiscible with one another, containing said active compound in said first liquid, confined within a hydrophilic solid shell layer.

67. The formulation according to claim 66, wherein said shell layer is configured to break or rupture under a mechanical load, particularly a manually applied mechanical load, more particularly by squeezing or chewing.
